(19) United States
(12) Patent Application Publication
(10) Pub. No.: US 2024/0321074 A1
(43) Pub. Date: Sep. 26, 2024

(54) DEVICE AND METHOD FOR DETERMINING SOIL MOISTURE

(71) Applicant: DRYAD NETWORKS GMBH, Eberswalde (DE)

(72) Inventors: Carsten BRINKSCHULTE, Berlin (DE); Marco BÖNIG, Dortmund (DE)

(73) Assignee: DRYAD NETWORKS GMBH, Eberswalde (DE)

(21) Appl. No.: 18/580,181

(22) PCT Filed: Jul. 13, 2022
(86) PCT No.: PCT/EP2022/069651

§ 371 Date: Jan. 18, 2024

(30) Foreign Application Priority Data
Jul. 19, 2021 (DE) 10 2021 118 588.2
Dec. 15, 2021 (DE) 10 2021 133 218.4

(51) Int. Cl. G08B 17/00; G01N 33/24; G08B 17/11; G08B 25/00

(52) U.S. Cl. CPC G08B 17/005; G01N 33/246; G08B 17/11; G08B 25/009

(57) ABSTRACT

The invention relates to a forest fire early detection system and/or forest fire risk analysis system with a sensor unit and an evaluation unit for analyzing the measured signals supplied by the sensor unit, the sensor unit having a signal source for emitting a signal, which signal source is suitable and intended for passing a signal into a nearby test specimen, as well as a method for forest fire early detection and/or forest fire risk analysis.

The invention relates to a forest fire early detection and/or forest fire risk analysis system comprising a sensor unit and an evaluation unit for evaluating the measured signals supplied by the sensor unit, as well as a method for forest fire early detection and/or forest fire risk analysis.

### PRIOR ART

Systems for early detection of forest fires are known. For this purpose, the area to be monitored is monitored using sensors. These sensors are, for example, rotatable cameras, but they have the disadvantage that they are less effective at night. Monitoring from a high orbit using an IR camera installed in a satellite has the disadvantage that the satellite is not geostationary, so it requires a certain amount of time to complete one orbit during which the area is not monitored. A satellite is also expensive to purchase, maintain and especially when launching the satellite. Monitoring by mini-satellites in low orbit usually requires a number of satellites, which are also expensive to launch. Satellite monitoring also involves high carbon dioxide emissions during launch. It makes more sense to monitor the area using a number of inexpensive, mass-produced sensors that work using optical smoke detection and/or gas detection. The sensors are distributed throughout the area and deliver data to a base station via radio connection.

Such a system for early detection of forest fires is presented in US 2008/0309502 A1. In the event of a fire alarm, a sensor delivers information to a nearby control terminal, which then triggers an alarm using a long-range radio frequency signal. This system has the disadvantage that the control terminal triggers the alarm and must have a powerful RF unit to do so. The sensors require a GPS unit that constantly sends a signal to the control terminal. Power consumption of the sensors is therefore high, and the service life of the sensors' energy sources (batteries) is limited.

It is therefore the object of the present invention to provide a forest fire early detection and/or forest fire risk analysis system that works reliably, can be expanded as desired and is inexpensive to install and maintain. It is also the object of the present invention to provide a method for forest fire early detection and/or forest fire risk analysis that works reliably, can be expanded as desired and is inexpensive to install and maintain. It is a further object of the present invention to provide a forest fire early detection and/or forest fire risk analysis terminal that works reliably and with sufficient precision and is inexpensive to install and maintain.

The stated object is achieved using the forest fire early detection and/or forest fire risk analysis system according to claim 1. Additional advantageous embodiments of the invention are set out in the dependent claims below.

The forest fire early detection and/or forest fire risk analysis system according to the invention has a sensor unit and an evaluation unit for evaluating the measured signals supplied by the sensor unit. In the international standard, the risk of forest fires is classified using a uniform warning level model with levels 1-5. In Germany, for example, the risk of forest fires is classified using the WBI forest fire risk index. In addition to atmospheric conditions such as temperature and humidity, the moisture of plants and/or the soil is also taken into account. While dry forest soil vegetation increases the risk of fire, green vegetation reduces the risk. The warning levels are primarily used to prevent forest fires.

The evaluation device for evaluating the measured signals supplied by the sensor unit is understood to mean at least one device which has an information input for accepting the measured signals from the sensor unit, an information processing unit for processing, in particular evaluating, the accepted measured signals, and an information output for passing on the processed and/or evaluated measured signals. The evaluation unit advantageously has components that include at least a processor, a memory, and an operating program with analysis and calculation routines. In particular, the electronic components of the evaluation device can be arranged on a circuit board (circuit board), preferably on a common circuit board with a control device, particularly preferably in the form of a microcontroller.

In addition, the control device and the evaluation device can particularly preferably also be designed as a single component. The evaluation device is provided to evaluate the measured signals received from the sensor unit and to determine at least one measured value from a sample. In addition, the evaluation and/or the sensor unit can have stored correction and/or calibration tables, which make it possible to interpret and/or convert and/or interpolate and/or extrapolate evaluation results and to calibrate the sensor unit and evaluation device.

According to the invention, the sensor unit has a signal source for emitting a signal. The signal source is intended and suitable for passing a signal into a nearby test specimen. The distance between the signal source and the test specimen is 0 centimeters, i.e., the signal source and the test specimen touch each other, up to a maximum of 10 meters. The signal source can emit a signal continuously, but it is preferred to emit signals at intervals.

According to the invention, the sensor unit has a detector unit for detecting a signal. The detector unit is intended and suitable for detecting a signal from a nearby test specimen. The distance between the detector unit and the test specimen is 0 centimeters, i.e. the detector unit and the test specimen touch each other, up to a maximum of 10 meters. The signal source can detect a detector unit continuously, but detection of signals at intervals is preferred.

In another embodiment of the invention, the forest fire early detection and/or forest fire risk analysis system has, a communication unit that is independent of the sensor unit in addition to the sensor unit. Using the communication unit, messages, in particular measurement data, are sent wirelessly as a data packet using a single-hop connection and/or a multi-hop connection.

In a further development of the invention, the sensor unit has a gas and/or temperature sensor. In addition to heavy smoke, a forest fire produces a variety of gases, particularly carbon dioxide and carbon monoxide. The type and concentration of these gases are characteristic of a forest fire and can be detected and analyzed using suitable sensors. The signals detected by the sensor unit are analyzed with regard to the concentration of the composition of the gases. If a concentration of the gases is exceeded, a forest fire is detected.

In addition, the temperature of the gases is analyzed. In addition to the type and concentration of the gases produced in a forest fire, their temperature is an indicator of a forest fire. The occurrence and/or presence of a forest fire is concluded by combining the analyzed concentrations of the composition of the gases and/or from the analyzed temperatures. The type, composition and temperature of the gases produced in a forest fire also indicate the occurrence of a forest fire. This makes it possible to detect an emerging forest fire and to combat it at an early stage.

In an advantageous embodiment of the invention, the sensor unit has a moisture sensor. Determining a moisture value means deriving statements from the backscattered wave trains obtained by the detection unit, which relate, among other things, to a relative and/or absolute moisture content and/or a moisture gradient.

In another design of the invention, the test specimen is the soil and/or an object in contact with the soil. The test specimen can also be a test specimen in the sense of a prototype. The test specimen then has specified properties such as shape, size or material composition like the soil. In particular, the test specimen has the same moisture value as the soil. In another embodiment, the test specimen can be the root of a tree.

In an advantageous embodiment of the invention, the signal comprises an acoustic and/or electrical signal and/or an electromagnetic wave with a wavelength range of 1 mm to 30 cm. Different methods can be used: An indirect method for determining the matrix potential is the gypsum block method. The electrical conductivity between two electrodes is measured as a function of the water content of the material in between. To prevent the influence of the fluctuating salt content in the soil, measurements are taken in the block in the saturated gypsum solution. However, the water content in the soil is not the same as in the gypsum block because there is a different capillary composition in the soil compared to gypsum. On the other hand, the solution in the block and the soil water are in equilibrium with respect to the matrix potential. The gypsum block must be calibrated specifically for the soil. Newer generations of gypsum block method sensors use tightly packed granules or ceramics that balance the moisture content of the soil.

The pF meter determines a water content of the test specimen. A sensor is connected to the soil matrix via a clay body. The clay body adapts to the matrix potential. The difference to conventional measuring methods is that the molar heat capacity is measured. The heat capacity changes linearly with the water content in the soil. Short heating pulses emitted by the signal source determine the heat capacity in the clay body and convert it to the applied matrix potential value using an internally stored calibration curve.

Time-domain reflectometry (TDR) determines the transit time of a pulse through electrode rods. This electromagnetic pulse depends on the dielectric constant of the medium surrounding the probe. For comparison, the speed of the pulse in a vacuum is equal to the speed of light. Ultrapure water has a permittivity of 78.38 As=Vm and soil between 3 and 5 As=Vm. The running time or capacity can be used to indirectly determine the water content of the soil matrix. Soil-specific calibration increases accuracy and reduces the influence of the soil structure. Temperature dependencies of the TDR measurement exist near the soil surface and in heavily clayey soils.

Ground Penetrating Radar (GPR) uses ultra-wideband technology to send very short pulses in the picosecond and nanosecond range into the ground. A separate antenna receives the transmitted and reflected signal. The speed and attenuation of the reflected signal can be used to determine the permittivity and conductivity and thus also the water content using the same analyses as with TDR. The GPR can be used to determine water content down to depths of up to 15 m. Similar methods include radar diffraction measurement, the passive microwave method and the electromagnetic induction method. The GPR and the methods mentioned are unsuitable for continuous measurements because they are fundamentally difficult to automate. Another effective method is the irradiation of sound waves into a test specimen, in particular ultrasonic waves with frequencies in the order of 20 KHz to 100 KHz. This takes advantage of the fact that the speed of the sound waves in the test specimen changes with the moisture content. In particular, a plurality of wave trains is passed into the soil, wherein the individual wave trains are transmitted continuously and/or at intervals by the signal source. In addition, capacitive sensors are used. A capacitive sensor is a sensor that works based on the change in electrical capacitance of a single capacitor or a system of capacitors. A capacitive sensor for measuring soil moisture, for example, consists of a plastic tube that is covered on the inside with two wide metal foils about 10 cm apart, the electrical capacitance of which is measured. This is strongly influenced by the permittivity of the environment, especially by the water content.

In another embodiment of the invention, the sensor unit has a detection unit, wherein the detection unit is suitable and intended to detect a return signal of the signal emitted by the sensor unit. The detection unit is further configured to detect an acoustic and/or electrical signal and/or an electromagnetic wave depending on the type of signal emitted. In a further development of the invention, the detection unit is suitable and provided for detecting a return signal of the signal passed into the sample body by the sensor unit.

In a further development of the invention, the detection unit is intended and suitable for detecting an acoustic and/or electrical signal and/or an electromagnetic wave in a wavelength range of 1 mm to 30 cm. The backscattered signal then also has the same wavelength range as the emitted signal.

In another embodiment of the invention, the forest fire early detection and/or forest fire risk analysis system comprises a gateway network that has a network server. The network also has multiple terminals. In such a network, one or more terminals are connected directly (single hub) to gateways via radio using LoRa modulation or FSK modulation FSK and communicate via the gateways with the Internet network server using a standard Internet protocol.

In a further development of the invention, the forest fire early detection and/or forest fire risk analysis system comprises a mesh gateway network which has a first gateway and a second gateway. The first and second gateways are combined in one device. These so-called mesh gateways are a combination of a first gateway and a second gateway. The mesh gateways communicate with each other using the MHF multi-hub wireless network, and at least one mesh gateway MGDn is connected to the network server via the standard Internet protocol.

In an advantageous embodiment of the invention, the first gateway communicates directly only with other gateways and terminals of the mesh gateway network. In particular, the communication between terminals and a first gateway is direct, i.e. without further intermediate stations (single-hop connection). Communication between the gateways can take place via a direct single-hop connection; a multi-hop connection is also possible. This simultaneously extends the range of the mesh gateway network because the first gateway is connected to the second gateway via a mesh multi-hop network and can therefore forward the data from the terminals to the Internet network server. The connection between the second gateway network server is wireless or wired.

In another embodiment of the invention, the mesh gateway network comprises an LPWAN and preferably a LoRaWAN. LPWAN describes a class of network protocols for connecting low-power devices such as battery-powered sensors to a network server. The protocol is designed in such a way that a long range and low energy consumption of the terminals can be achieved with low operating costs. LoRaWAN requires particularly little energy. The LoRaWAN networks implement a star-shaped architecture using gateway message packets between the terminals and the central network server. The gateways are connected to the network server, while the terminals communicate with the respective gateway via LoRa.

In another embodiment of the invention, the second gateway has a communication interface that provides an Internet connection to the network server. The Internet connection is a wireless point-to-point connection, preferably using a standard Internet protocol.

In another embodiment of the invention, the terminals and/or the first gateways have a self-sufficient energy supply. To be able to install and operate the terminals and the first gateways connected thereto even in inhospitable and especially rural areas far from energy supplies, the terminals and the first gateways are equipped with a self-sufficient energy supply. The energy supply can be provided, for example, by energy stores—also rechargeable ones.

In a further development of the invention, the self-sufficient energy supply has an energy store and/or an energy conversion device. In particular, energy supply using solar cells should be mentioned, in which energy conversion from light to electrical energy takes place. The electrical energy is usually stored in an energy store in order to ensure the energy supply even in times of low solar radiation (e.g. at night).

In another embodiment of the invention, the terminals and the first gateways are operated off-grid. Due to the self-sufficient energy supply of terminals and first gateways, these devices can be operated autonomously without a supply network. Therefore, terminals and first gateways can be distributed and networked, particularly in impassable areas that cannot be reached with conventional radio networks.

The task is further solved using the method for forest fire early detection and/or forest fire risk analysis. Other embodiments of the invention are set out in the dependent claims. The method for forest fire early detection and/or forest fire risk analysis has four method steps: In the first method step, a signal is emitted by a signal source of the sensor unit. The signal can emitted continuously or preferably at intervals. In the second method step, the signal is passed into a nearby test specimen. The passing can take place by directly connecting the signal source to the test specimen or via a suitable line. The test specimen is therefore arranged at a distance of 0 m to 10 m from the signal source. In the third method step, a signal is detected with a detection unit of the sensor unit. In the fourth method step, the detected signal is evaluated. In particular, the evaluation includes a classification of the forest fire risk using a risk level system. In addition, a forest fire that has already broken out can be detected.

In a further development of the invention, the detected signal is a backscattered signal of the emitted signal. The signal backscattered on a test specimen therefore allows conclusions to be drawn about the risk of forest fires.

In another embodiment of the invention, the gas composition and/or the temperature is determined from the detected signal. In addition to heavy smoke, a forest fire produces a variety of gases, particularly carbon dioxide and carbon monoxide. The type and concentration of these gases are characteristic of a forest fire and can be detected and analyzed using suitable sensors. The signals detected by the sensor unit are analyzed with regard to the concentration of the composition of the gases. If a concentration of the gases is exceeded, a forest fire is detected. In addition, the temperature of the gases is analyzed. In addition to the type and concentration of the gases produced in a forest fire, their temperature is an indicator of a forest fire. The occurrence and/or presence of a forest fire is concluded by combining the analyzed concentrations of the composition of the gases and/or from the analyzed temperatures. The type, composition and temperature of the gases produced in a forest fire also indicate the occurrence of a forest fire.

In another advantageous embodiment of the invention, the moisture of the test specimen is determined from the detected signal. The backscattered wave train statements received from the detection unit are evaluated to determine a relative and/or absolute moisture content and/or a moisture gradient of the test specimen.

In another embodiment of the invention, the test specimen is the soil and/or an object in contact with the soil. The moisture of the soil is determined. The test specimen can also be a test specimen in the sense of a prototype. The test specimen then has specified properties such as shape, size or material composition like the soil. In particular, the test specimen has the same moisture value as the soil. In another embodiment, the test specimen can be the root or the stem of a tree.

In another embodiment of the invention, an acoustic and/or electrical signal and/or an electromagnetic wave with a wavelength range of 1 mm to 30 cm is emitted. For example, the gypsum block method, a pF meter, time domain reflectometry (TDR), the irradiation of radar or sound waves and/or the use of capacitive sensors or a combination of the options mentioned are used.

In a further development of the invention, an acoustic and/or electrical signal and/or an electromagnetic wave in a wavelength range of 1 mm to 30 cm is detected. The backscattered signal then also has a same wavelength range as the emitted signal. The detection unit is configured to detect an acoustic and/or electrical signal and/or an electromagnetic wave depending on the type of signal emitted.

In another embodiment of the invention, the method is carried out using a forest fire early detection and/or forest fire risk analysis system. The forest fire early detection and/or forest fire risk analysis system comprises a gateway network with a network server and multiple terminals, wherein the sensor unit is part of a terminal and the signals and/or the evaluated signals are transmitted to the network server via the gateway. In such a network, one or more terminals are connected directly (single hub) to gateways via radio using LoRa modulation or FSK modulation FSK and communicate via the gateways with the Internet network server using a standard Internet protocol.

In another embodiment of the invention, the forest fire early detection and/or forest fire risk analysis system has a mesh gateway network with a first gateway and a second gateway, wherein the evaluated signals are transmitted to the network server via the first gateway and the second gateway. This achieves an extension of the range of LoRaWAN networks by interposing the multi-hop network using gateways and thus maintaining full compatibility with the LoRaWAN specification.

In another embodiment of the invention, the first gateway communicates directly only with other gateways and terminals of the mesh gateway network, and the second gateway communicates with the network server. In particular, the communication between terminals and a first gateway is direct, i.e. without further intermediate stations (single-hop connection). Communication between the gateways can take place via a direct single-hop connection; a multi-hop connection is also possible. This simultaneously extends the range of the mesh gateway network because the first gateway is connected to the second gateway via a mesh multi-hop network and can therefore forward the data from the terminals to the Internet network server. The connection between the second gateway network server is wireless or wired.

In another embodiment of the invention, the communication of the mesh gateway network takes place via an LPWAN and preferably a LoRaWAN protocol. The first gateway is connected to the second gateways via the meshed multi-hop radio network, and the data from the terminals is forwarded to the Internet network server. This removes the range limitation of the direct connection between terminals and gateways provided for by the LoRaWAN standard.

In another embodiment of the invention, the terminals and/or the first gateways are supplied with energy via a self-sufficient energy supply. To be able to install and operate the terminals and the first gateways connected thereto even in inhospitable and especially rural areas far from energy supplies, the terminals and the first gateways are equipped with a self-sufficient energy supply. The energy supply can be provided, for example, by energy stores—also rechargeable ones.

In a further development of the invention, the self-sufficient energy supply has an energy store and/or an energy conversion device. In particular, energy supply using solar cells should be mentioned, in which energy conversion from light to electrical energy takes place. The electrical energy is usually stored in an energy store to ensure energy supply even in times of low solar radiation (e.g. at night).

In another embodiment of the invention, the terminals and the first gateways are operated off-grid. Due to the self-sufficient energy supply of terminals and first gateways, these devices can be operated autonomously without a supply network. Therefore, terminals and first gateways can be distributed and networked, particularly in impassable areas that cannot be reached with conventional radio networks.

The object is also achieved using the forest fire early detection and/or forest fire risk analysis terminal. Other embodiments of the invention are set out in the dependent claims. The forest fire early detection and/or forest fire risk analysis terminal according to the invention has a signal source for emitting a signal, a detection unit for detecting a signal, and a communication unit. The emitted signal can be emitted continuously or preferably at intervals. The emitted signal is an acoustic and/or electrical signal and/or an electromagnetic wave with a wavelength range of 1 mm to 30 cm. The detection unit is configured to detect an acoustic and/or electrical signal and/or an electromagnetic wave with a wavelength range of 1 mm to 30 cm. Using the communication unit, messages, in particular measurement data, can be sent wirelessly as a data packet using a single-hop connection and/or a multi-hop connection.

In a further development of the invention, the communication unit is arranged separately from the signal source and the detection unit. The signal source and detection unit can be connected to the communication unit, for example via a cable connection or Bluetooth connection, such that the signal source and detection unit can also be flexibly arranged at a distance from the communication unit.

Embodiments of the forest fire early detection and/or forest fire risk analysis system according to the invention, the method according to the invention for forest fire early detection and/or forest fire risk analysis, and the forest fire early detection and/or forest fire risk analysis terminal according to the invention are shown schematically in simplified form in the drawings and are explained in more detail in the following description.

Wherein:

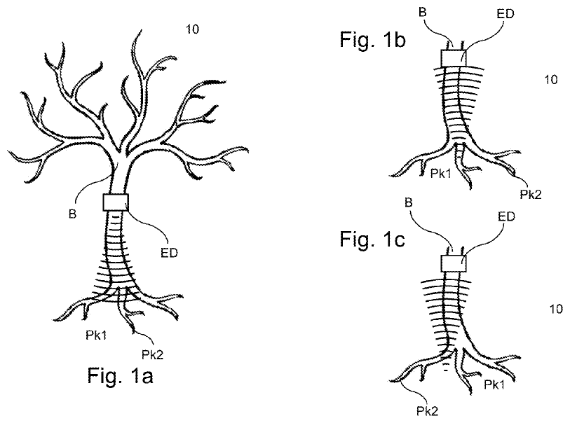

FIG. 1 a: shows the emission of a wave by the forest fire early detection and/or forest fire risk analysis system according to the invention

FIG. 1 b: shows the detection of a wave backscattered from a root by the forest fire early detection and/or forest fire risk analysis system

FIG. 1 c: shows the detection of a wave backscattered from the forest soil by the forest fire early detection and/or forest fire risk analysis system

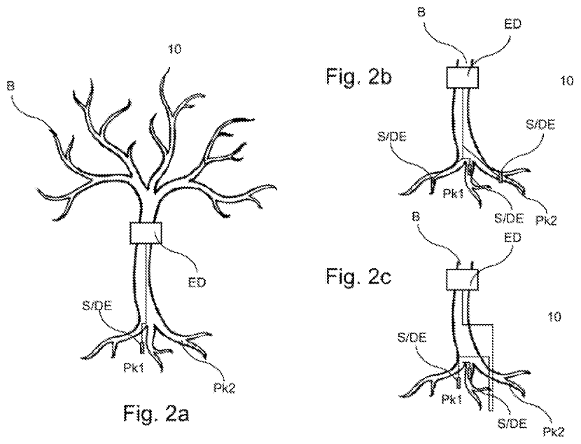

FIG. 2 a: shows a sensor/detector unit connected to a forest fire early detection and/or forest fire risk analysis terminal in contact with the forest soil

FIG. 2 b: shows multiple sensor/detector units connected to the forest fire early detection and/or forest fire risk analysis terminal in contact with the tree roots shows two sensor/detector units connected to a forest fire early detection FIG. 2 c; and/or forest fire risk analysis terminal in contact with the tree root and the forest soil

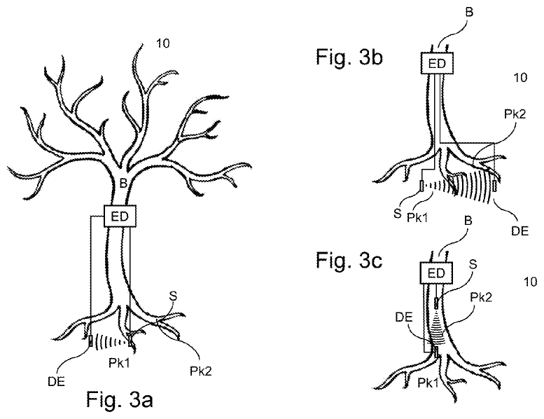

FIG. 3 a: shows a sensor unit and detection unit of a forest fire early detection and/or forest fire risk analysis system

FIG. 3 b: shows a sensor unit and detection unit of a forest fire early detection and/or forest fire risk analysis system coupled to the tree stem

FIG. 3 c: shows a sensor unit and detection unit of a forest fire early detection and/or forest fire risk analysis system coupled to the soil

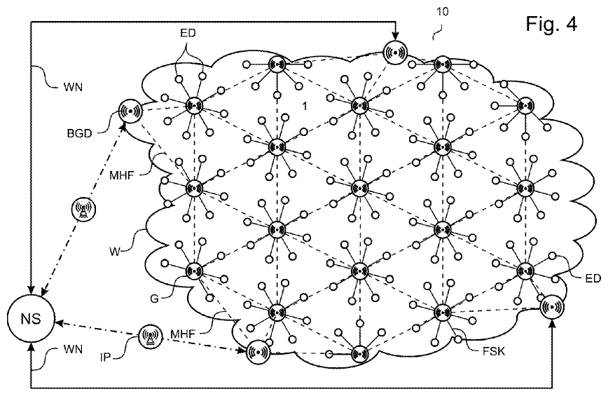

FIG. 4: shows a LoRaWAN mesh gateway network with terminals, a network server, gateways and border gateways

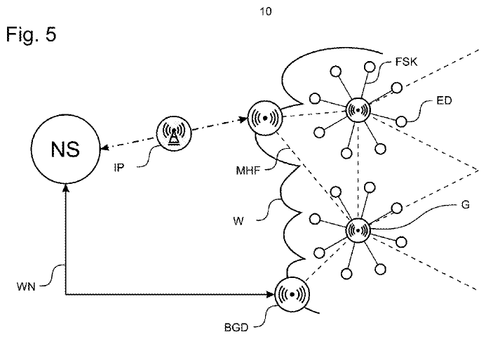

FIG. 5: shows a detailed view of the forest fire early detection and/or forest fire risk analysis system according to the invention

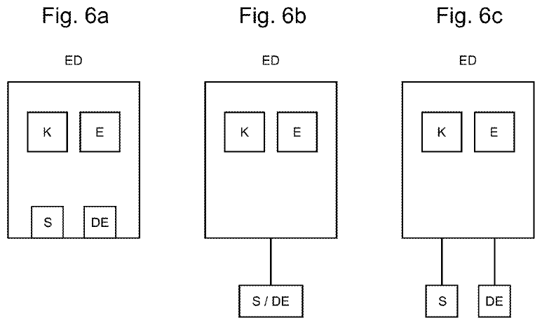

FIG. 6 a: shows exemplary embodiments of the forest fire early detection and/or forest fire risk analysis terminal

FIG. 6 b: shows exemplary embodiments of the forest fire early detection and/or forest fire risk analysis terminal

FIG. 6 c: shows exemplary embodiments of the forest fire early detection and/or forest fire risk analysis terminal

FIG. 1 shows an exemplary embodiment of the forest fire early detection and/or forest fire risk analysis system 10 according to the invention. Sensor unit SE with signal source S and detection unit DE are arranged in the forest fire early detection and/or forest fire risk analysis terminal ED. The forest fire early detection and/or forest fire risk analysis terminal ED itself is arranged on a tree B at a distance from the forest soil, which forms a test specimen PK1.

To determine the forest fire risk or a forest fire, the signal source S arranged in the terminal ED sends a signal into the test specimens PK1, PK2 (FIG. 1 a). In this exemplary embodiment, the first test specimen PK1 is the forest soil, the second test specimen PK2 is a root of the tree B. The emitted signal is backscattered from the test specimens PK1, PK2 (FIG. 1 b, 1 c) and detected by the detection unit DE, which is also arranged in the terminal ED. The signal emitted is an acoustic, an electrical, and/or an electromagnetic signal. If the signal is a wave, the wave has a wavelength of 1 mm to 30 cm. The signal detected by the detection unit DE then also has a wavelength of 1 mm to 30 cm.

A moisture value of the test specimens PK1, PK2 is then determined from the backscattered signal using the evaluation unit. The evaluation unit can be arranged in the terminal ED itself; the moisture value is then transmitted via a gateway network 1 or a mesh gateway network 1 (see FIG. 4) to the network server NS and stored there. The evaluation unit can also be arranged externally, preferably on the network server NS (see FIG. 4). In this case, only the backscattered signal is transmitted to the network server NS using a gateway network 1 or a mesh gateway network 1. The evaluation unit also determines a moisture value. In this exemplary embodiment, the determined moisture value is an average value of the test specimens PK1, PK2 (forest soil and tree roots).

Another exemplary embodiment of the forest fire early detection and/or forest fire risk analysis system 10 according to the invention is shown in FIG. 2. In this exemplary embodiment, in contrast to the previous exemplary embodiment (see FIG. 1), no average moisture value of the test specimens PK1, PK2, but rather a moisture value for each test specimen PK1, PK2 is determined. In this exemplary embodiment, capacitive sensors are preferably used, which are arranged in the test specimens PK1, PK2. A capacitive sensor is a sensor that works based on the change in electrical capacitance of a single capacitor or a system of capacitors. The sensor should first be calibrated on the ground, ideally on site, to achieve high accuracy.

The forest fire early detection and/or forest fire risk analysis terminal ED is arranged on a tree B at a distance from the forest soil. Sensor unit SE with signal source S and detection unit DE are arranged in a device and connected to the forest fire early detection and/or forest fire risk analysis terminal ED by means of a cable connection. A plurality of sensor units SE connected to the terminal ED can also be arranged in such a way that the sensor unit SE is arranged in the forest soil PK1 (FIG. 2 a), at different locations of the root PK2 of the tree B (FIG. 2 b), or in the forest soil PK1 and at the root PK2 (FIG. 2 c). Any combination of the arrangements mentioned is also possible. The evaluation unit advantageously determines a moisture value for each sensor unit SE, in this exemplary embodiment a moisture value for the forest soil PK1 (FIG. 2 a), an average moisture value for the roots PK2 of the tree B (FIG. 2 b), and an average moisture value for the forest soil PK1 and a root PK2 of tree B (FIG. 2 c).

FIG. 3 shows another exemplary embodiment of the forest fire early detection and/or forest fire risk analysis system 10 according to the invention. In this exemplary embodiment, the sensor unit SE is divided in such a way that the signal source S and detection unit DE are at a distance from one another and each of them is connected via a cable connection to the forest fire early detection and/or forest fire risk analysis terminal ED. Due to the distance from the signal source S to the detection unit DE on the one hand and the signal source S or detection unit DE to the terminal ED on the other hand, a flexible arrangement of the forest fire early detection and/or forest fire risk analysis system 10 is possible; in addition, moisture values can be determined from different test specimens.

Signal source S and detection unit DE are arranged in such a way that they conduct a signal through the forest soil PK1 (FIG. 3 a). A moisture value of the forest soil PK1 is therefore determined using the evaluation unit. In addition, the signal source S and the detection unit DE can be arranged at such a distance from one another that an average value of the moisture of two test specimens PK1, PK2 is determined (FIG. 3 b). Signal source S and detection unit DE can also be arranged in such a way that the test specimen PK2 is the trunk of tree B (FIG. 3 c). For this purpose, the signal source S emits an electromagnetic signal in the range of 1 cm (centimeter waves), which has a penetration depth of approx. 15 cm into the wood. The signal emitted by the signal source S therefore penetrates through the tree bark into the tree trunk. An average value of the moisture value of the tree trunk PK2 is therefore determined using the evaluation unit. In addition, the terminal ED can optionally have a temperature sensor and/or a gas sensor. The gas composition and/or temperature is determined from the detected signal.

An exemplary embodiment of a LoRaWAN mesh gateway network 1 according to the invention as part of the forest fire early detection and/or forest fire risk analysis system 10 is shown in FIG. 4. The LoRaWAN mesh gateway network 1 has a mesh gateway network 1 that utilizes the technology of a LoRaWAN network. The LoRaWAN network has a star-shaped architecture in which message packets are exchanged between the sensors ED and a central Internet network server NS by means of gateways.

The LoRaWAN mesh gateway network 1 has a large number of sensors ED, which are connected to gateways G via a single-hop connection FSK. The gateways G are usually mesh gateways MGD. The mesh gateways MGD are connected to each other and partly to border gateways BGD. The border gateways BGD are connected to the Internet network server NS, either via a wired connection WN or via a wireless connection using the Internet protocol IP.

A detailed view of a forest fire early detection system 10 according to the invention is shown in FIG. 5. The forest fire early detection system 10 has a plurality of terminals ED equipped with sensors, with eight terminals ED each communicating with a gateway G via a single-hop connection FSK. The FGD gateways are connected to each other and to BGD border gateways. The border gateways BGD are connected to the Internet network server NS, either via a wired connection WN or via a wireless connection using the Internet protocol IP.

FIG. 6 shows three variants of an exemplary embodiment of a forest fire early detection and/or forest fire risk analysis terminal ED. To be able to install and operate the ED terminal in inhospitable and especially rural areas far away from energy supplies, the ED terminal is equipped with a self-sufficient energy supply E. In the simplest case, the energy supply E is a battery, which can also be designed to be rechargeable. However, the use of capacitors, especially supercapacitors, is also possible. The use of solar cells is somewhat more complex and cost-intensive, but offers a very long service life for the terminal ED. In addition to the energy supply E, a memory and power electronics (not shown) are also arranged in the terminal ED.

In addition, a terminal ED has the signal source S, which emits an acoustic and/or electrical signal and/or an electromagnetic wave with a wavelength range of 1 mm to 30 cm. The detection unit DE is configured to receive a backscattered signal. The sensor ED also has the communication interface K. Using the communication interface K, messages from the terminal ED, in particular measurement data, are sent as a data packet wirelessly to a gateway G, MDG, BDG using a single-hop connection FSK via LoRa (chirp frequency spread modulation) or frequency modulation.

All of the components mentioned are arranged in a housing to protect them from the effects of the weather (FIG. 6 a). Signal source S and detection unit DE can also be connected to the terminal ED via a cable connection, wherein signal source S and detection unit DE can be arranged in a housing (FIG. 6 b) or separately from one another (FIG. 6 c). A combination of the above-mentioned arrangements of signal source S and detection unit DE is also possible. An evaluation unit is arranged in the network server NS, but it may also be arranged in the terminal ED.

### LIST OF REFERENCE NUMERALS

1 LoRaWAN mesh gateway network
10 forest fire early detection and/or forest fire risk analysis system
ED forest fire early detection and/or forest fire risk analysis terminal/terminal
G gateways
NS Internet network server
IP Internet protocol
W forest
B tree
MHF multi-hub wireless network
BGD border gateway
FSK FSK modulation
WN wired connection
SE sensor unit
S signal source
DE detection unit
K communication unit of the terminal
E energy supply
EK energy conversion device
ES energy store
PK, PK1, PK2 test specimen

1. A forest fire early detection and/or forest fire risk analysis system (10), having
a sensor unit (SE)
an evaluation unit for evaluating the measured signals supplied by the sensor unit (SE),
characterized in that
the sensor unit (SE) has a signal source (S) for emitting a signal, which signal source is suitable and intended to pass a signal into a nearby test specimen (PK, PK1, PK2).

2. The forest fire early detection and/or forest fire risk analysis system (10) according to claim 1,
characterized in that
the forest fire early detection and/or forest fire risk analysis system (10) has a communication unit (K) that is independent of the sensor unit (SE), in addition to the sensor unit (SE).

3. The forest fire early detection and/or forest fire risk analysis system (10) according to claim 1,
characterized in that
the sensor unit (SE) has a gas sensor and/or a temperature sensor.

4. The forest fire early detection and/or forest fire risk analysis system (10) according to claim 1,
characterized in that
the sensor unit (SE) has a moisture sensor.

5. The forest fire early detection and/or forest fire risk analysis system (10) according to claim 1,
characterized in that
the test specimen (PK, PK1, PK2) is the soil and/or an object in contact with the soil.

6. The forest fire early detection and/or forest fire risk analysis system (10) according to claim 1,
characterized in that the signal comprises an acoustic and/or electrical signal and/or an electromagnetic wave with a wavelength range of 1 mm to 30 cm.

7. The forest fire early detection and/or forest fire risk analysis system (10) according to claim 1,
characterized in that the sensor unit (SE) has a detection unit (DE), wherein the detection unit (DE) is suitable and intended to detect a return signal of the signal emitted by the sensor unit (SE).

8. The forest fire early detection and/or forest fire risk analysis system (10) according to claim 7,
characterized in that the detection unit (DE) is intended and suitable for detecting an acoustic and/or electrical signal and/or an electromagnetic wave in a wavelength range of 1 mm to 30 cm.

9. The forest fire early detection and/or forest fire risk analysis system (10) according to claim 1,
characterized in that the forest fire early detection and/or forest fire risk analysis system (10) has a gateway network (1) with a network server (NS) and multiple terminals (ED).

10. The forest fire early detection and/or forest fire risk analysis system (10) according to claim 9,
characterized in that
the forest fire early detection and/or forest fire risk analysis system (10) has a mesh gateway network (1) with a first gateway (G1) and a second gateway (G2).

11. The forest fire early detection and/or forest fire risk analysis system (10) according to claim 10,
characterized in that
the first gateway (G1) communicates directly with other gateways (G1, G2) and terminals (ED) of the mesh gateway network (1) only, and the second gateway (G2) communicates with the network server (NS).

12. The forest fire early detection and/or forest fire risk analysis system (10) according to claim 10,
characterized in that
the mesh gateway network (1) comprises an LPWAN and preferably a LoRaWAN.

13. The forest fire early detection and/or forest fire risk analysis system (10) according to claim 10,
characterized in that
the second gateway (G2) has a communication interface (K) that provides an Internet connection (IP) to the network server (NS).

14. The forest fire early detection and/or forest fire risk analysis system (10) according to claim 10,
characterized in that
the terminals (ED) and/or the first gateways (G1) have a self-sufficient energy supply (E).

15. The forest fire early detection and/or forest fire risk analysis system (10) according to claim 14,
characterized in that
the self-sufficient energy supply (E) comprises an energy store (ES) and/or an energy conversion device (EK).

16. The forest fire early detection and/or forest fire risk analysis system (10) according to claim 10,
characterized in that
the terminals (ED) and the first gateways (G1) are operated off-grid.

17. A method for forest fire early detection and/or forest fire risk analysis with the method steps
Emitting a signal from a signal source (S) of the sensor unit (SE)
Passing the signal into a nearby test specimen (PK, PK1, PK2)
Detecting a signal with a detection unit (DE) of the sensor unit (SE)
Evaluating the (detected) signal

18. The method for forest fire early detection and/or forest fire risk analysis according to claim 17,
characterized in that
the detected signal is a backscattered signal of the emitted signal.

19. The method for forest fire early detection and/or forest fire risk analysis according to claim 17,
characterized in that
the gas composition and/or temperature is determined from the detected signal.

20. The method for forest fire early detection and/or forest fire risk analysis according to claim 17,
characterized in that
the moisture of the test specimen (PK1, PK2) is determined from the detected signal.

21. The method for forest fire early detection and/or forest fire risk analysis according to claim 17,
characterized in that
the test specimen (PK, PK1, PK2) is the soil and/or an object in contact with the soil, wherein the moisture of the soil is determined.

22. The method for forest fire early detection and/or forest fire risk analysis according to claim 17,
characterized in that
an acoustic and/or electrical signal and/or an electromagnetic wave with a wavelength range of 1 mm to 30 cm is emitted.

23. The method for forest fire early detection and/or forest fire risk analysis according to claim 17,
characterized in that
an acoustic and/or electrical signal and/or an electromagnetic wave with a wavelength range of 1 mm to 30 cm is detected.

24. The method for forest fire early detection and/or forest fire risk analysis according to claim 17,
characterized in that
the method is carried out using a forest fire early detection and/or forest fire risk analysis system (10),
wherein the forest fire early detection and/or forest fire risk analysis system (10) comprises a gateway network (1) with a network server (NS) and multiple terminals (ED),
wherein the sensor unit (SE) is part of a terminal (ED) and the signals and/or the evaluated signals are transmitted via the gateway (G1, G2) to the network server (NS).

25. The method for forest fire early detection and/or forest fire risk analysis according to claim 24,
characterized in that
the forest fire early detection and/or forest fire risk analysis system (10) has a mesh gateway network (1) with a first gateway (G1) and a second gateway (G2),
wherein the evaluated signals are transmitted via the first gateway (G1) and the second gateway (G2) to the network server (NS).

26. The method for forest fire early detection and/or forest fire risk analysis according to claim 24,
characterized in that
the first gateway (G1) communicates directly with other gateways (G1, G2) and terminals (ED) of the mesh gateway network (1) only, and the second gateway (G2) communicates with the network server (NS).

27. The method for forest fire early detection and/or forest fire risk analysis according to claim 24,
characterized in that
the communication of the mesh gateway network (1) takes place via an LPWAN and preferably a LoRaWAN protocol.

28. The method for forest fire early detection and/or forest fire risk analysis according claim 17,
characterized in that
the terminal (ED) and/or the first gateways (G1) are supplied with energy via a self-sufficient energy supply (E).

29. The method for forest fire early detection and/or forest fire risk analysis according to claim 28,
characterized in that
the self-sufficient energy supply (E) comprises an energy store (ES) and/or energy conversion device (EK).

30. The method for forest fire early detection and/or forest fire risk analysis according to claim 17,
characterized in that
the terminals (ED) and the first gateways (G1) are operated off-grid.

31. A forest fire early detection and/or forest fire risk analysis terminal (ED) having
a signal source (S) for emitting a signal,
a detection unit (DE) for detecting a signal,
a communication unit (K).

32. The forest fire early detection and/or forest fire risk analysis terminal (ED) according to claim 31,
characterized in that
the communication unit (K) is arranged separately from the signal source (S) and the detection unit (DE).
